(19) United States
(12) Patent Application Publication
(10) Pub. No.: US 2018/0207332 A1
(43) Pub. Date: Jul. 26, 2018

(54) MEDICAL DEVICES AND RELATED METHODS

(71) Applicant: Boston Scientific Scimed, Inc., Maple Grove, MN (US)

(72) Inventors: Kenneth P. Reever, Hopedale, MA (US); William Stanhope, Lunenburg, MA (US); Eric Wong, South Grafton, MA (US)

(73) Assignee: Boston Scientific Scimed, Inc., Maple Grove, MN (US)

(21) Appl. No.: 15/876,400

(22) Filed: Jan. 22, 2018

Related U.S. Application Data
(60) Provisional application No. 62/449,151, filed on Jan. 23, 2017.

(51) Int. Cl. A61M 1/00

(52) U.S. Cl. CPC A61M 1/0058; A61M 1/0011; A61M 1/0017

(57) ABSTRACT

A medical system includes an irrigation system including an irrigation bag containing irrigation fluid and an irrigation line, and a medical device with at least two internal lumens connecting at least two ports on a handle to a distal end of the medical device. The medical system also includes a suction system including a suction source, a waste container, and a suction line, and a controller that controls at least one of the irrigation system and the suction system. The irrigation line is coupled to a first port, and the suction line is coupled to a second port.

### CROSS-REFERENCE TO RELATED APPLICATIONS

This patent application claims the benefit of priority under 35 U.S.C. § 119 to U.S. Provisional Patent Application No. 62/449,151, filed Jan. 23, 2017, which is herein incorporated by reference in its entirety.

### TECHNICAL FIELD

Various aspects of the present disclosure relate generally to medical systems and related methods. More specifically, the present disclosure relates to medical systems and methods for providing irrigation and suction in a medical procedure.

### BACKGROUND

Pressurized irrigation and suction have been used in a wide variety of medical procedures to control or manage the fluid flow and internal pressure in a body cavity of a patient. During a procedure, applying suction allows a user to remove unwanted material. Irrigation may also be applied in order to maintain an internal pressure within the body cavity to assist in visibility as the user performs a procedure. However, fluid management systems include complicated pumps, pressure sensors, stopcocks, and elaborate tubing sets. These fluid management systems are generally expensive, complex, and single-use. Moreover, the suction pressure provided by wall-mounted suction ports or outlets may be unknown or may vary greatly between different hospitals or facilities.

The systems, devices, and methods of the current disclosure may rectify some of the deficiencies described above, and/or address other aspects of the prior art.

### SUMMARY

Examples of the present disclosure relate to, among other things, medical systems. Each of the examples disclosed herein may include one or more of the features described in connection with any of the other disclosed examples.

In one example, a medical system may include an irrigation system including an irrigation bag containing irrigation fluid and an irrigation line, and a medical device with at least two internal lumens connecting at least two ports on a handle to a distal end of the medical device. The medical system may also include a suction system including a suction source, a waste container, and a suction line, and a controller that controls at least one of the irrigation system and the suction system. The irrigation line may be coupled to a first port, and the suction line may be coupled to a second port.

The system may further include one or more of the following features. The controller may control the suction system by controlling the opening and/or closing of a pinch valve. The medical system may further include a foot pedal that controls the opening and/or closing of the pinch valve. The irrigation bag may be supported by a stand at a height above the medical device. The height may be adjustable. The medical system may also include a pressure cuff surrounding the irrigation bag. The controller may control a pressure of the pressure cuff. The controller may control the pressure of the pressure cuff and/or a suction pressure of the suction system to maintain a desired pressure differential.

The medical system may further include an irrigation load cell and a suction load cell. The irrigation load cell may be coupled to the irrigation system to provide a value indicative of the mass of the irrigation bag, and the suction load cell may be coupled to the suction system to provide a value indicative of the mass of the suction waste container. The irrigation load cell and suction load cell may be coupled to the controller, and the controller may control a suction pressure of the suction system based the values of the irrigation load cell and the suction load cell.

The irrigation system may further include a pinch valve to open and/or close the irrigation line. The controller may further include a display and an interface to indicate values associated with the irrigation pressure and/or the suction pressure. The controller may further include an air compressor to control the pressure of the irrigation system, and the suction source may be a suction pump within the controller to control the pressure of the suction system. The suction source may be a wall-mounted suction source.

In another example, a medical system may include a medical device, a controller, an irrigation system including an irrigation source and an irrigation line coupled to the medical device, and a suction system including a suction line coupled to the medical device. The controller may include at least one pressure sensor to detect the pressure within the irrigation line or within the suction line.

The system may further include one or more of the following features. The at least one pressure sensor may detect the pressure within the irrigation system, and the controller may control the pressure of the irrigation source. The at least one pressure sensor may detect the pressure within the suction line, and the controller may control the pressure of the suction line.

In another example, a medical device controller may include a first scale, a second scale, and a suction source with adjustable suction pressure. The medical device controller may adjust the suction pressure of the suction source as a function of values of the first scale and the second scale.

The device may further include one or more of the following features. The first scale may measure a mass of a waste container, and the second scale may measure a mass of an irrigation bag. The controller may further include a display to display the measured values associated with the first scale and the second scale.

Both the foregoing general description and the following detailed description are exemplary and explanatory only and are not restrictive of the features, as claimed. As used herein, the terms “comprises,” “comprising,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” is used herein in the sense of “example,” rather than “ideal.” As used herein, the terms “about,” “substantially,” and “approximately,” indicate a range of values within +/−5% of a stated value.

### BRIEF DESCRIPTION OF THE DRAWINGS

The accompanying drawings, which are incorporated in and constitute a part of this specification, illustrate exemplary features of the present disclosure and together with the description, serve to explain the principles of the disclosure.

FIG. 1 illustrates an exemplary medical system;

FIG. 2 illustrates a medical system according to another aspect of the disclosure;

FIG. 3 illustrates a medical system according to another aspect of the disclosure; and

FIG. 4 illustrates a medical system according to another aspect of the disclosure.

### DETAILED DESCRIPTION

Examples of the present disclosure relate to medical systems for delivering irrigation and suction during a medical procedure. The medical systems may include any appropriate insertion device to deliver treatment to a bodily orifice.

Reference will now be made in detail to examples of the present disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device or insertion device. When used herein, “proximal” refers to a position relatively closer to the exterior of the body or closer to an operator using the medical device or insertion device. In contrast, “distal” refers to a position relatively further away from the operator using the medical device or insertion device, or closer to the interior of the body.

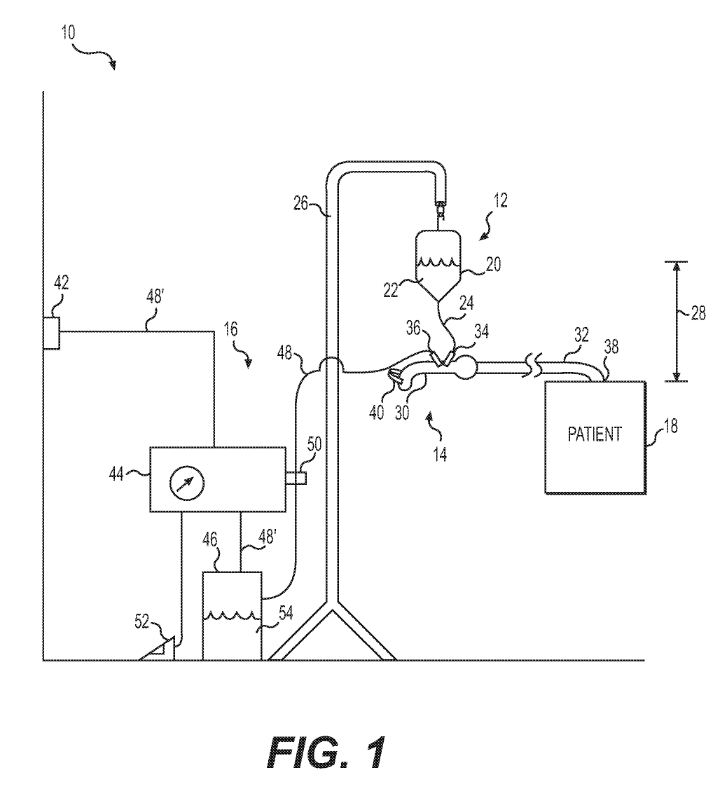

FIG. 1 illustrates a medical system 10 that includes an irrigation system 12, a medical device 14, and a suction system 16. Medical device 14 may be inserted into a body cavity 18 of a patient to perform medical treatment. Body cavity 18 refers to any internal lumen of a patient. Irrigation system 12 may be connected to the medical device 14 to deliver irrigation to the body cavity 18. Suction system 16 may also be connected to the medical device 14 to deliver suction to the body cavity 18. A user may control the irrigation system 12 and/or the suction system 16 to control the visibility and/or pressure within the body cavity 18.

As shown in FIG. 1, irrigation system 12 includes an irrigation source, such as, for example, irrigation bag 20, irrigation fluid 22, and an irrigation line 24 connecting the irrigation bag 20 to the medical device 14. Irrigation bag 20 may be supported by a stand 26, which may be an IV pole that is adjustably lockable to different heights or include a plurality of hooks at varying heights to allow the user to adjust the height of the irrigation bag 20. In one instance, the user may adjust stand 26 to allow the user to position the irrigation bag 20 at varying heights 28 above the medical device 14 or the body cavity 18. Although not shown, irrigation system 12 may include a control valve on irrigation line 24 to control the flow irrigation fluid 22. Irrigation bag 20 may be an intravenous fluid bag or other irrigation vessel. Irrigation fluid 22 may be any medically safe surgical fluid, such as, for example, saline solution. Irrigation line 24 may be a single-use, disposable tubing cassette or other appropriate medical tubing.

Medical device 14 may include a handle 30 and a shaft 32. Handle 30 may include a plurality of ports, including at least an irrigation port 34 and a suction port 36. Shaft 32 may include a plurality of internal lumens (not shown) connecting the ports to a distal end 38 of shaft 32 such that, when the shaft 32 is be inserted into body cavity 18, a user may deliver medical treatment via handle 30 through the irrigation port 34 and suction port 36. Handle 30 may include a deflection lever 40 at a proximal end of the handle 30 to deflect the distal end 38 of shaft 32 and steer the shaft 32 in the body cavity 18. Handle 30 may also include a visualization mechanism (not shown), which may include a power source, a camera positioned at distal end 38 of shaft 32, and a display. Medical device 14 may include additional ports through which medical treatment may be delivered. Medical device 14 may be a ureteroscope, a hysteroscope, a resectoscope, a cystoscope, or any similar endoscope device.

Suction system 16 may include a suction source 42, a controller 44, a waste container 46, and at least one suction line 48. In one aspect, the suction source 42 may be a wall-mounted suction source (FIG. 1), the suction pressure of which may vary greatly depending on the facility. Controller 44 may regulate the suction pressure from suction source 42, and may include a pinch valve 50 and an actuator or switch, such as, for example, a foot pedal 52. Although not shown, controller 44 may be powered by being plugged into a power outlet or by an internal battery. Waste container 46 may be a sealed container. Suction line 48 may be a single-use, disposable tubing cassette. Suction line 48 may be coupled to medical device 14 via suction port 36 in handle 30. Suction line 48 may be threaded through pinch valve 50 and may terminate in waste container 46, which collects waste fluid 54. An additional suction line 48′ may connect the suction source 42 to the controller 44 and waste container 46. Foot pedal 52 may be electronically connected to controller 44 and may be operable to allow the user to controllably open the pinch valve 50, and thus control the suction pressure through the suction line 48. Alternatively, a control button (not shown) may be positioned on handle 30 and may control the pinch valve 50 to control the suction pressure.

During use, a user may use handle 30 to position medical device 14 such that distal end 38 of shaft 32 is within the body cavity 18 of a patient. The user may lockably adjust stand 26 to position irrigation bag 20 to be a height 28 above the body cavity 18 to adjust the irrigation pressure applied by irrigation system 12. The height 28 determines a pressure differential between the irrigation bag 20 and the body cavity 18, so no irrigation fluid 22 flows through the irrigation line 24 into body cavity 18 when the pressure within the body cavity 18 is static. For example, if suction is not being applied by suction system 16, and irrigation fluid 22 has pressurized body cavity 18 to a pressure equal to the irrigation pressure determined by height 28, then no irrigation fluid 22 would flow through irrigation line 24. Additionally, if the body cavity 18 has a pressure greater than the irrigation pressure determined by height 28, then no irrigation fluid 22 would flow through irrigation line 24. However, if the body cavity 18 has a pressure less than the irrigation pressure determined by height 28, then irrigation fluid 22 would flow through irrigation line 24 until body cavity 18 reaches a pressure equal to the irrigation pressure determined by height 28.

The user may also selectively activate the suction system 16 through action on the foot pedal 52. The user may utilize the medical device 14 to perform a medical treatment at the treatment site within body cavity 18, which may include, for example, cutting or otherwise removing tissue. Therefore, the suction system 16 may aid in evacuating the body cavity 18 to help remove unwanted material that may occlude the user's view or interfere with treatment of the treatment site, such as, for example, tissue or blood. As the suction system 16 evacuates the body cavity 18, irrigation fluid 22 may flow through the irrigation line 24 and into body cavity 18 to fill the body cavity 18 and regulate the internal pressure within the body cavity 18. The pressure and rate at which the irrigation fluid 22 flows into the body cavity 18 may depend on the suction pressure of suction system 16 and the height 28 the irrigation bag 20 is above body cavity 18. The user may deactivate the suction system 16 by removing pressure on foot pedal 52 to close the pinch valve 50, and irrigation fluid 22 may continue to flow from the irrigation bag 20 into body cavity 18 until a pressure equilibrium is established.

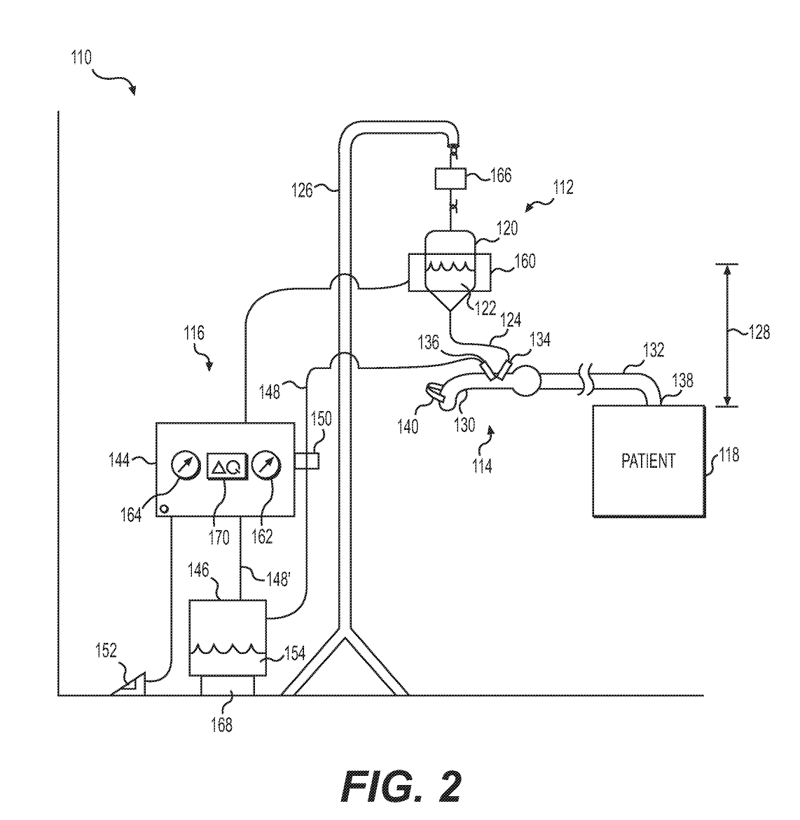

FIG. 2 illustrates an alternative example with similar elements to the medical system 10 shown by 100 added to the reference numbers. This aspect illustrates medical system 110 with an irrigation system 112 similar to the gravity assisted irrigation system 12 of FIG. 1 with an additional pressure source, such as, for example, a pressure cuff 160 on irrigation bag 120. Pressure cuff 160 may be coupled to controller 144 such that a user may control both the irrigation system 112 and the suction system 116 from the same controller. Therefore, medical system 110 may function similar to medical system 10 described above, but irrigation system 112 may introduce irrigation fluid 122 from irrigation bag 120 through irrigation line 124 under greater pressure than with a height differential from height 128 alone.

Moreover, controller 144 may control an air compressor, which may be internal or external, to inflate and control the air pressure of the pressure cuff 160 around irrigation bag 120. Controller 144 may control the suction pressure from a suction source (not shown) as discussed with respect to FIG. 1, or controller 144 may control a suction pump, which may be internal or external, to provide suction for suction system 116. Controller 144 may further include irrigation control 162 and suction control 164 to allow the user to control the irrigation system 112 and the suction system 116. Irrigation control 162 and suction control 164 may be adjustable dials or knobs with markings corresponding to various pressure selections, or may be any appropriate user interface.

Additionally, medical system 110 may include at least two load cells electronically and/or wirelessly coupled to the controller 144. An irrigation load cell 166 may be positioned above the irrigation bag 120 to measure the mass of the irrigation bag 120 and irrigation fluid 122. A waste load cell 168 may be positioned beneath the waste container 146 to measure the mass of the waste container 146 and the collected waste fluid 154. The controller 144 may include electronics and/or software to calculate fluid loss or gain internal to the body cavity 118 by comparing the difference between the values of irrigation load cell 166 and waste load cell 168. The controller 144 may display a fluid loss or gain on a display 170.

Controller 144 may also include electronics and/or software that are programmed to control the pinch valve 150 and/or other valves to control the fluid flow through irrigation system 112 and suction system 116. Foot pedal 152 may control the fluid flow or pressure of irrigation system 112, or foot pedal 152 may control the pinch valve 150 to control the suction of suction system 116. Controller 144 of medical system 110 may be powered by an internal battery source, or medical system 110 may include an electrical connection to a wall outlet (not shown).

In use, a user may insert medical device 114 into body cavity 118 as previously discussed. The user may also manipulate the irrigation control 162 to set the air pressure on pressure cuff 160, and may manipulate the suction control 164 to set the suction pressure through suction line 148. The user may apply pressure to the foot pedal 152 to open the pinch valve 150, applying suction to body cavity 118 and reducing the pressure within body cavity 118. As a result, the irrigation fluid 122 will flow from irrigation bag 120 into body cavity 118 through irrigation line 124 and an internal lumen in medical device 114 because the pressure within body cavity 118 is lower than the pressure within the irrigation bag 120. Controller 144, through a sensor on pressure cuff 160, may then detect a drop in volume of irrigation fluid 122 in irrigation bag 120 based on a drop in the pressure in the pressure cuff 160. The electronics and/or programming of controller 144 may the activate the air compressor to return pressure cuff 160 to the pressure level set by irrigation control 162. As a result, controller 144 ensures that body cavity 118 remains at an appropriate pressure even though suction system 116 removes fluid from body cavity 118. When the user releases pressure on foot pedal 152 to cease suction, irrigation system 112 may continue to deliver irrigation fluid 122 to body cavity 118 until the pressure of pressure cuff 160 matches the pressure within body cavity 118, reaching pressure equilibrium.

During the above procedure, controller 144 may detect and present on display 170 a change in fluid within body cavity 118. Through irrigation load cell 166, controller 144 may detect a change in mass, and thus change in volume, of irrigation fluid 122 in irrigation bag 120. Through suction load cell 168, controller 144 may detect a change in mass, and thus change in volume, of waste fluid 154 collected in waste container 146. If the controller 144 detects that a greater volume of waste fluid 154 has been collected than the volume of irrigation fluid 122 delivered, the controller 144 may display that difference on display 170. As such, the user may increase the pressure of pressure cuff 160 through irrigation control 162, or may reduce the suction through suction line 148 through suction control 164. In an opposite situation, the user may reduce the pressure of pressure cuff 160 through irrigation control 162, or may increase the suction through suction line 148 through suction control 164. As such, the user may fine-tune the pressure and suction to control the pressure within body cavity 118. Alternatively, the controller 144 may include internal electronics and/or programming that automatically adjust irrigation control 162 and/or suction control 164 to control irrigation system 112 and/or suction system 116 to maintain an appropriate pressure within body cavity 118.

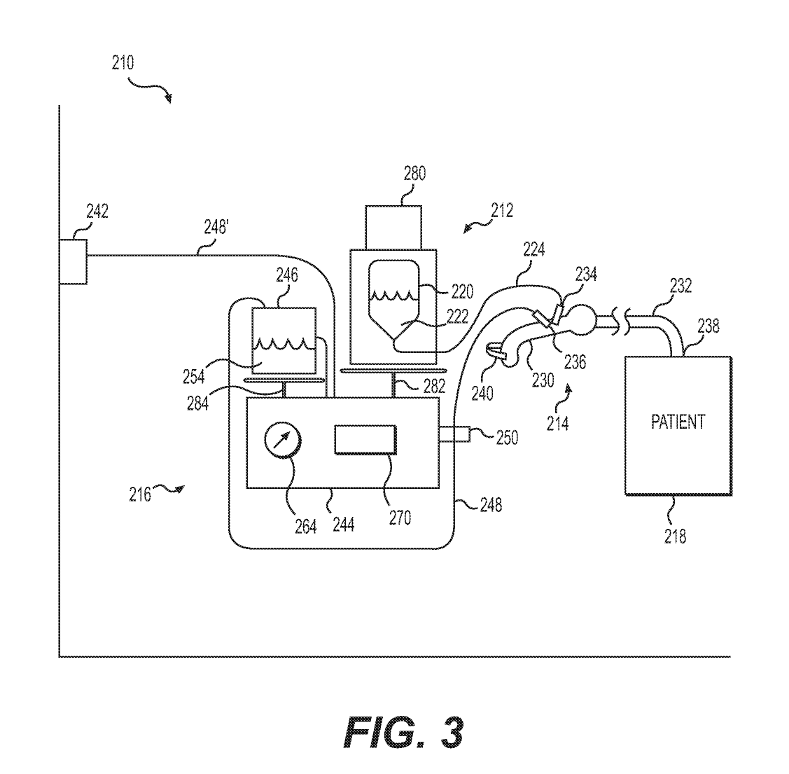

Referring to FIG. 3, which is an alternative example with similar elements to the medical system 10 shown by 200 added to the reference numbers, medical system 210 may allow a user to determine a difference between delivered irrigation fluid 222 and removed waste fluid 254. Medical system 210 includes an irrigation system 212 with an irrigation bag 220 containing irrigation fluid 222 and an irrigation line 224 connected to an irrigation port 234 on handle 230 of medical device 214. Irrigation bag 220 may be positioned without a stand, and pressure on irrigation bag 220 may be provided by placing a static weight 280 on irrigation bag 220. Weight 280 may be between approximately 5 and 20 pounds, and weight 280 may be one weight or may comprised of a plurality of weights.

As shown, irrigation bag 220 may be positioned on an irrigation scale 282 rather than mounted on a stand. Similarly, waste container 246 may be positioned on a waste scale 284. Irrigation scale 282 and waste scale 284 may be integral with controller 244, or may be separate from but electronically connected to controller 244. Controller 244 may include a display 270 that may display masses or volumes of the collected waste fluid 254 and delivered irrigation fluid 222, or a difference between the collected waste fluid 254 and delivered irrigation fluid 222. Controller 244 may also include additional controls to tare and/or reset the display 270.

Controller 244 may control the suction pressure from a suction source 242 as discussed with respect to FIG. 1, or controller 244 may control a suction pump, which may be internal or external, to provide suction for suction system 216. Controller 244 may further include suction control 264 to allow the user to set and control the suction system 216. For example, the user may set the suction control 264 at the beginning of a procedure, and/or may later adjust the suction control 264 based on the difference between the collected waste fluid 254 and delivered irrigation fluid 222 displayed on display 270. Alternatively, controller 244 may include internal electronics and/or programming that automatically adjusts the suction control 264 to control the suction system 216 to maintain an appropriate pressure within the body cavity 218 based on the difference between the collected waste fluid 254 and delivered irrigation fluid 222 displayed on display 270.

In use, a user may position the medical device 214 within body cavity 218 and may set the suction pressure with suction control 264 of controller 244. The user may activate the suction through pinch valve 250 or any appropriate control, including a foot pedal, a control button on the medical device 214, or a control button on controller 244. As suction system 216 removes fluid from body cavity 218, the weight 280 applies a constant pressure to irrigation bag 220 to fill body cavity 218 with irrigation fluid 222. Display 270 may provide a difference between collected waste fluid 254 measured by waste scale 284 and delivered irrigation fluid 222 measured by irrigation scale 284. The user may manually, or the controller 244 may automatically, adjust the suction control 264 on controller 244 based on any fluid difference displayed on display 270. Alternatively, the user may adjust the pressure on irrigation bag 220 by increasing or decreasing the mass of weight 280. As such, the pressure and fluid within body cavity 218 may be monitored and stabilized so the user may perform medical treatment within body cavity 218.

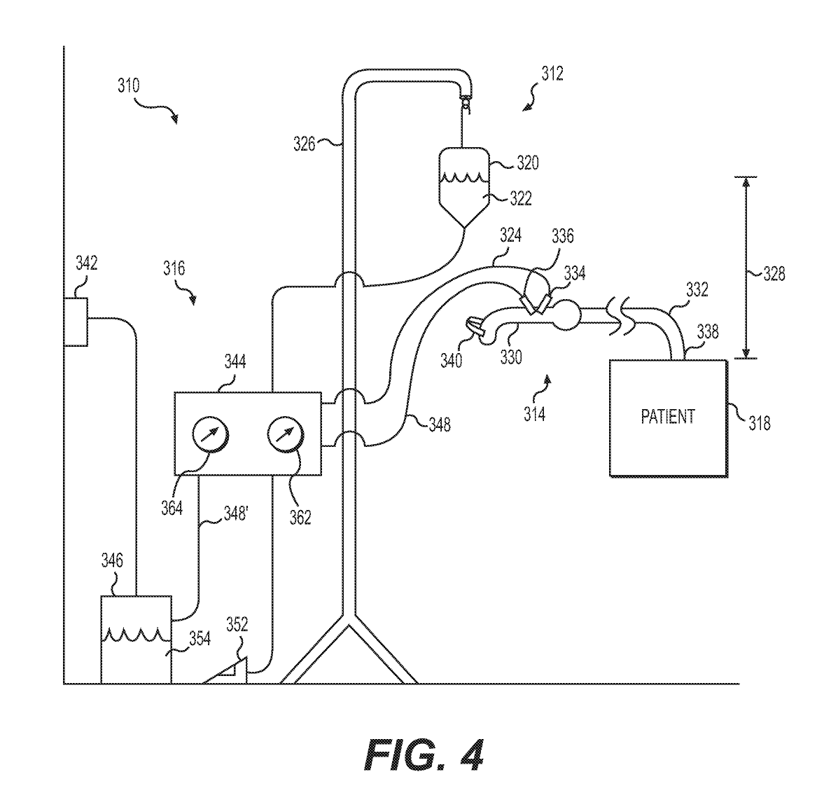

Referring to FIG. 4, which is an alternative example with similar elements to the medical system 10 shown by 300 added to the reference numbers, medical system 310 may allow a user to set and control the pressure of irrigation system 312 and suction of suction system 316. As shown, irrigation bag 320 may be supported by stand 326 and connected to irrigation port 334 of medical device 314 by irrigation line 324, with irrigation line 324 passing through controller 344. Irrigation bag 320 may be positioned a height 328 above body cavity 318, or irrigation bag 320 be positioned proximate to controller 344 without stand 326. Controller 344 may include an air compressor to impart additional pressure to irrigation system 312, and maybe controlled by irrigation control 362. As such, controller 344 may add pressure in addition to any height 328 difference between the irrigation bag 320 on stand 326, or controller 344 may be the sole source of pressure in irrigation system 312.

Controller 344 may also include suction control 364 to allow the user to set and control the suction system 316, which may further include suction source 342, waste container 346, suction line 348, and foot pedal 352. Controller 344 may include internal pressure sensors to measure the respective pressures through the irrigation line 324 and the suction line 348. As such, user activation, through foot pedal 352 or another actuator, may initiate suction through suction line 348 at the set suction pressure, as well as backfilling the body cavity 318 with irrigation fluid 322 at the set irrigation pressure.

Controller 344 may be powered by a connection to a wall power outlet (not shown), for example, through foot pedal 352. Although not shown, both suction irrigation system 312 and suction system 316 may include pinch valves to control fluid flow.

In another aspect, irrigation system 312 may be controlled by the foot pedal 352, and controller 344 may impart a standard suction pressure. In this example, irrigation control 362 and suction control 364 may instead indicate the instantaneous respective pressures.

Furthermore, controller 344 may be used to set a desired pressure differential between the irrigation line 324 and the suction line 348, and may indicate the respective pressures on a display (not shown). In this aspect, the foot pedal 352 may control the irrigation system 312. The controller 344 may detect a difference from the desired pressure differential and may activate the suction system 316 to control the pressure within the body cavity 318.

Controller 344 may also be used to control the irrigation system 312 to maintain a set pressure differential. Here, the foot pedal 352 may control the suction system 316. Activating the suction system 316 would lower the pressure within body cavity 318. Controller 344 may detect a difference in the pressure differential between the irrigation line 324 and the suction line 348, and, as a result, controller 344 may open the irrigation line 324 to refill and restore the pressure within the body cavity 318.

The disclosed medical systems 10, 110, 210, and 310 control the irrigation and suction through a medical device, and may help enable reliable, safe, and inexpensive medical procedures. For example, a user may perform an endoscopic procedure in a body cavity, applying suction and backfilling with irrigation, without using stopcocks, pumps, or motors. A user may use a wall-mounted suction source, even though the suction pressure between different wall-mounted suction sources may vary, because the controller regulates the suction pressure applied through the medical device. Alternatively, the controller may include its own suction source, allowing for greater mobility. Moreover, a user may use an irrigation bag and irrigation line without the risk of over pressurizing the irrigation bag, irrigation line, or the body cavity because the pressure is limited by the bag's height, the pressure cuff, the weight on the bag, and/or the pressure differential between the body cavity and the irrigation bag caused by the suction system. As such, a user may perform a medical procedure within a body cavity, remove unwanted material that may occlude the procedure, and irrigate the body cavity in order to maintain an appropriate pressure and visibility within the body cavity.

While principles of the present disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

We claim:

1. A medical system, comprising:
an irrigation system including an irrigation bag containing irrigation fluid and an irrigation line;
a medical device with at least two internal lumens connecting at least two ports on a handle to a distal end of the medical device;
a suction system including a suction source, a waste container, and a suction line; and
a controller that controls at least one of the irrigation system and the suction system,
wherein the irrigation line is coupled to a first port, and the suction line is coupled to a second port.

2. The medical system of claim 1, wherein the controller controls the suction system by controlling the opening and/or closing of a pinch valve.

3. The medical system of claim 2, further comprising a foot pedal that controls the opening and/or closing of the pinch valve.

4. The medical system of claim 1, wherein the irrigation bag is supported by a stand at a height above the medical device.

5. The medical system of claim 4, wherein the height is adjustable.

6. The medical system of claim 1, further comprising a pressure cuff surrounding the irrigation bag.

7. The medical system of claim 6, wherein the controller controls a pressure of the pressure cuff.

8. The medical system of claim 7, wherein the controller controls the pressure of the pressure cuff and/or a suction pressure of the suction system to maintain a desired pressure differential.

9. The medical system of claim 1, further comprising an irrigation load cell and a suction load cell.

10. The medical system of claim 9, wherein the irrigation load cell and suction load cell are coupled to the controller;
wherein the irrigation load cell is coupled to the irrigation system to provide a value indicative of the mass of the irrigation bag, and wherein the suction load cell is coupled to the suction system to provide a value indicative of the mass of the suction waste container; and
wherein the controller controls a suction pressure of the suction system based on the values of the irrigation load cell and the suction load cell.

11. The medical system of claim 10, wherein the irrigation system further comprises a pinch valve to open and/or close the irrigation line.

12. The medical system of claim 1, wherein the controller further comprises a display and an interface to indicate values associated with the irrigation pressure and/or the suction pressure.

13. The medical system of claim 12, wherein the controller further comprises an air compressor to control the pressure of the irrigation system; and
wherein the suction source is a suction pump within the controller to control the pressure of the suction system.

14. The medical system of claim 1, wherein the suction source is a wall-mounted suction source.

15. A medical system, comprising:
a medical device;
a controller;
an irrigation system including an irrigation source and an irrigation line coupled to the medical device; and
a suction system including a suction line coupled to the medical device;
wherein the controller includes at least one pressure sensor to detect the pressure within the irrigation line or within the suction line.

16. The medical system of claim 15, wherein the at least one pressure sensor detects the pressure within the irrigation system; and
wherein the controller controls the pressure of the irrigation source.

17. The medical system of claim 15, wherein the at least one pressure sensor detects the pressure within the suction line; and
wherein the controller controls the pressure of the suction line.

18. A medical device controller, comprising:
a first scale;
a second scale; and
a suction source with adjustable suction pressure;
wherein the medical device controller adjusts the suction pressure of the suction source as a function of values of the first scale and the second scale.

19. The medical device controller of claim 18, wherein the first scale measures a mass of a waste container, and the second scale measures a mass of an irrigation bag.

20. The medical device controller of claim 19, wherein the controller further includes a display to display the measured values associated with the first scale and the second scale.
